(19) United States
(12) Patent Application Publication
(10) Pub. No.: US 2004/0001884 A1
(43) Pub. Date: Jan. 1, 2004

(54) Pharmaceutical tablet coating composition

(75) Inventors: .Antonio Moroni, Morris Plains, NJ (US); William J. Drefko, Kearny, NJ (US)

Correspondence Address: INTERNATIONAL SPECIALTY PRODUCTS, Attn: William J. Davis, Esq., Legal Department, Building No. 10, 1361 Alps Road, Wayne, NJ, 07470, US

(73) Assignee: ISP INVESTMENTS INC.

(21) Appl. No.: 10/177,516

(22) Filed: Jun. 21, 2002

(51) Int. Cl. A61K 9/20; A61K 9/36

(52) U.S. Cl. 424/465; 424/480; 523/122

(57) ABSTRACT

A pharmaceutical tablet coating composition which is a synergistic combination of hydroxypropylmethyl cellulose and polyvinylpyrrolidone-vinyl acetate copolymer, providing coatings having high gloss, substantially no color fading for pigmented tablets and superior tensile strength.

### BACKGROUND OF THE INVENTION

1. Field of the Invention

This invention relates to pharmaceutical tablet coatings, and, more particularly, to coating compositions which provide coatings having a high gloss, no color fading and high tensile strength.

2. Description of the Prior Art

There are many reasons for coating pharmaceutical tablets; some aesthetic, some functional. One important reason is to enhance drug stability; that is, to protect the drug from oxygen, moisture and light, the three key causes of drug degradation. Coatings can also be used to separate reactive components in a tablet formulation.

Another important reason for tablet coating is identification. Tablet coatings, or films thereon, may take on a variety of colors. A coated tablet may also be imprinted with a symbol or word. In the case of the film-coated tablet, the tablet core may be embossed with a symbol or word that remains visible after the coating process. The definitive identification of a coated tablet has saved patients and health care professionals alike. Additionally, coatings are used to uniquely identify a branded product.

Tablet coating is done for aesthetic reasons as well. Often the appearance of the tablet core is mottled or otherwise unattractive. A coating with high gloss masks this.

A film-coated tablet consists of a core around which a thin, colored polymer film is deposited; thus, a film-coated tablet gains about 3% of total tablet weight upon coating.

The basic film coating formula consists of a film former, a pigment dispersion, a plasticizer, and a solvent. A variety of polymeric film formers have been used to coat tablets. The solubility properties of the polymer can produce an immediate-release, an enteric-release, or a sustained-release tablet, as desired.

Suitable immediate-release film formers are water-soluble cellulose ether polymers, for example, hydroxypropylmethylcellulose (HPMC). Low viscosity grades of these polymers have been employed in the coating formulation to maximize polymer solids concentration.

However, for one or more reasons, HPMC alone does not possess the optimum attributes for tablet coating. Accordingly, it is an object of this invention to provide new and improved tablet coating compositions which can provide superior tablet coatings.

### SUMMARY OF THE INVENTION

What is described herein is a pharmaceutical tablet coating composition comprising a synergistic combination of (a) hydroxypropylmethylcellulose (HPMC) and (b) a polyvinylpyrrolidone (VP)-vinyl acetate (VA) copolymer, preferably wherein (b) is present in a weight amount of 5-45%, and most preferably, wherein (b) is 10-20%. Preferably, the copolymer has a 60:40 wt. ratio of VP and VA monomers.

The tablet coating compositions of the invention provide coatings having high gloss, substantially no color fading for pigmented tablets, and superior tensile strength, with desirable composition viscosity properties.

### EXAMPLE 1

### (Clear Films)

Table 1 below is a summary of experimental results comparing a composition of the immediate-release film-former HPMC, alone or with polyethylene glycol (PEG 400), as additive therein, with compositions of (a) HPMC and, as additive, (b) a copolymer of vinylpyrrolidone (PVP) and vinyl acetate (VA) (International Specialty Products) (S630-60:40 wt. % ratio of monomers), present in a w/w amount of 10-20% in the composition.

The results show that clear films (without pigment) incorporating S630 as the additive with HPMC have higher tensile strengths than compositions of HPMC alone or with PEG 400 as the additive. PEG 400 substantially decreases the tensile strength of HPMC while S630 has an elevating effect. Films using S630 also have similar elasticity to HPMC while those incorporating PEG 400 show a much increased elasticity at the expense of tensile strength.

TABLE 1

| | | | Coating (Clear Films) | | | | | | |
|---|---|---|---|---|---|---|---|---|---|
| Composition | | | | | Minimum | | | % | Mean |
| | | % | Length | Thickness | x-sectional | Force | Stretch | Elonga- | Tensile |
| Resin | Additive | Additive | (mm) | (mm) | area | (g) | (mm) | tion | Strength (g/mm²) (ave.) |
| HPMC | S630 | 20 | 98.0 | 0.030 | 0.38 | 2095.5 | 1.680 | 1.70 | |
| HPMC | S630 | 10 | 97.5 | 0.030 | 0.38 | 2219 | 1.460 | 1.50 | |
| | | | | | | | | | 6219.5 |
| HPMC | S630 | 10 | 99.0 | 0.051 | 0.65 | 4300 | 2.320 | 2.34 | |
| HPMC | S630 | 15 | 100 | 0.056 | 0.71 | 2489.3 | 1.900 | 1.90 | |
| HPMC | PEG 400 | 20 | 99.6 | 0.117 | 1.49 | 158.3 | 7.980 | 8.01 | |
| HPMC | PEG 400 | 15 | 99.6 | 0.066 | 0.838 | 480.8 | 9.320 | 9.36 | |
| HPMC | PEG 400 | 15 | 99.6 | 0.114 | 1.45 | 567.5 | 6.730 | 6.76 | |
| HPMC | PEG 400 | 10 | 99.6 | 0.071 | 0.903 | 356.8 | 7.620 | 7.65 | |
| | | | | | | | | | 409.5 |
| HPMC | PEG 400 | 10 | 99.6 | 0.086 | 1.1 | 466.9 | 9.960 | 10.0 | |
| HPMC | — | 0 | 100.0 | 0.041 | 0.52 | 2990 | 2.565 | 2.60 | |
| HPMC | — | 0 | 100.0 | 0.051 | 0.64 | 3370 | 5.000 | 5.00 | |
| HPMC | — | 0 | 100.1 | 0.051 | 0.64 | 3890 | 2.803 | 2.80 | |
| | | | | | | | | | 5439.2 |
| HPMC | — | 0 | 100.5 | 0.051 | 0.64 | 3410 | 2.513 | 2.50 | |
| HPMC | — | 0 | 100.3 | 0.071 | 0.9 | 3870 | 4.073 | 4.10 | |
| HPMC | — | 0 | 99.8 | 0.043 | 0.55 | 3250 | 3.263 | 3.30 | |

Tests performed on Texture Technologies TAXT2i Texture Analyzer with 5 kg load
Test Parameters: measure force in tension distance 10.0 mm
hold until time time 90 s
test speed 0.1 mm/s trigger force 2 g
Sample prep: films laid on glass with gardner knife set to 30 mills dried 24 hours at 50° C. conditioned 2 hours at 23° C., 40% RH cut with razor blade and template to dumbbell shape 100 mm long, 30 mm base width, 12.7mm minimum width
Tensile Strength TS = peak force/minimum cross sectional area

### EXAMPLE 2

### (Pigment Films)

The gloss characteristics of pigmented HPMC film coatings, alone and with the addition of S630, were evaluated. The test samples consisted of coatings of a 2 kg charge of embossed placebo. Formulations were as follows.

Pigment Dispersion

60% Dl water

20% Red # 40 High Dye Lake or Blue #2

20% Titanium Dioxide

This pigment dispersion was used in combination with the test resin systems. Systems containing HPMC were modified with PEG 400 at 15% of the HPMC solids, and “resin solids” were calculated using HPMC only. The test system contained 10% S630 as additive. All coatings were completed at 12% solids.

| Coating Conditions | |
|---|---|
| Spray rate: | 20 g/min |
| Inlet air temp: | 70° +/31 2°C. |
| Exhaust air temp: | 48° +/31 2°C. |
| Atomizing air pressure: | 40 psi |
| Pan RPM: | 13 +/−2 |
| Process air volume: | 230 CFM +/31 10 CFM |

The gloss meter used was the surface analysis system 805A (Tricor).

Reported gloss measurements were the top 10% of pixels measured for each sample.

The results in Table 2 below also shows that the invention compositions (with pigment Red #40, 50%, TiO₂ 50%) shows advantageously higher gloss than the standard (PEG 400 at 15%) both at lower and higher S630 levels.

TABLE 2
(PIGMENTED FILMS)

| | | | Resin/Pigment | |
|---|---|---|---|---|
| Resin | Additive | Additive % | (wt/wt) | Gloss* |
| HPMC | PEG 400 | 15 | 2:1 | 139 |
| HPMC | PEG 400 | 15 | 2.5:1 | 137 |
| HPMC | PEG 400 | 15 | 3:1 | 139 |
| HPMC | S630 | 10 | 2:1 | 145 |
| HPMC | 5630 | 10 | 2.5:1 | 145 |
| HPMC | S630 | 10 | 3:1 | 150 |
| HPMC | S630 | 20 | 2:1 | 142 |
| HPMC | S630 | 20 | 2.5:1 | 138 |
| HPMC | S630 | 20 | 3:1 | 132 |
| HPMC | S630 | 40 | 2:1 | 136 |
| HPMC | S630 | 40 | 3:1 | 144 |

*The results show that the S630 additive composition had higher gloss levels than the PEG 400 system.

The invention compositions had a lower viscosity than the standard. This lowering of viscosity significantly reduces the need for atomization air pressure which is beneficial for gloss improvement. With standardized parameters, the reduction in air pressure from 40 to 35 psi produces higher increase in gloss values, in formulations containing S630 rather than PEG 400, as shown in Table 3.

TABLE 3

| | | | Air | Resin/ | | |
|---|---|---|---|---|---|---|
| | | | Pressure, | Pigment | Viscosity* | |
| Resin | Additive | Additive | psi % | (wt/wt) | (sec) | Gloss |
| HPMC | S630 | 40 | 40 | 3:1 | 12.5 | 144 |
| HPMC | S630 | 40 | 35 | 3:1 | 12.5 | 155 |
| HPMC | PEG 400 | 15 | 40 | 3:1 | 17 | 139 |
| HPMC | PEG 400 | 15 | 35 | 3:1 | 17 | 144 |

*Zahn cup #3

In addition, advantageously the invention compositions (with pigment) did not fade at all while the standard faded appreciably, especially for pastel color coatings. Stability tests were carried out either at room temperature (RT) or under accelerated conditions (AC), i.e. 40° C./75% RH.

Color changes were measured using the L,a,b color space model using Delta E (numerical absolute volume of the color differences) calculated from each sample L,a,b values measured at timed intervals.

TABLE 4
Evaluation of Fading in Films
Containing S630 and PEG 400
(Pigment Added at 40% of HPMC)

| | | | | Change in Color | |
|---|---|---|---|---|---|
| | | | | (Fading) After 4 | |
| | | | | Weeks (ΔE) | |
| | | | | Ageing | |
| | | % | | Conditions | |
| Resin | Additive | (wt/wt) | Pigment | RT | AC |
| HPMC | S630 | 10 | Blue #2, 2%, | .15 | 1.89 |
| | | | TiO₂ 98% | | |
| HPMC | S630 | 20 | Blue #2, 2%, | .25 | 1.96 |
| | | | TiO₂ 98% | | |
| HPMC | PEG 400 | 10 | Blue #2, 2%, | 1.95 | 9.80 |
| | | | TiO₂ 98% | | |
| HPMC | PEG 400 | 20 | Blue #2, 2%, | 8.15 | 18.15 |
| | | | TiO₂ 98% | | |
| HPMC | PEG 400 | 10 | Red #40, 50%, | .68 | 1.71 |
| | | | TiO₂ 50% | | |
| HPMC | S630 | 10 | Red #40, 50%, | .4 | 1.13 |
| | | | TiO₂ 50% | | |

While the invention has been described with particular reference to certain embodiments thereof, it will be understood that changes and modifications may be made which are within the skill of the art. Accordingly, it is intended to be bound only by the following claims, in which:

What is claimed is:

1. A pharmaceutical tablet coating composition comprising a synergistic combination of (a) hydroxypropylmethylcellulose (HPMC), and (b) a polyvinylpyrrolidone (VP)-vinyl acetate (VA) copolymer.

2. A composition according to claim 1 wherein (b) is present in a weight amount of 5-45% in said composition.

3. A composition according to claim 2 wherein said amount is 10-20%.

4. A composition according to claim 1 wherein said copolymer has a 60:40 wt. ratio of monomers.

5. A solution of the composition of claim 1.

6. A pharmaceutical tablet coated with the composition of claim 1.

7. A pharmaceutical tablet according to claim 6 whose coating has a high gloss, substantially no color fading, and a high tensile strength.
